(19) United States
(12) Patent Application Publication
(10) Pub. No.: US 2022/0134048 A1
(43) Pub. Date: May 5, 2022

(54) SYSTEMS AND METHODS FOR VIRTUAL-REALITY ENHANCED QUANTITATIVE MEDITATION

(71) Applicant: Sensei Wellness Holdings, Inc., Santa Monica, CA (US)

(72) Inventors: Daniel GRUNEBERG, Santa Monica, CA (US); Andy BAUCH, Santa Monica, CA (US); Brian PASS, Santa Monica, CA (US); Danny TRINH, Santa Monica, CA (US); Addison KOWALSKI, Santa Monica, CA (US)

(21) Appl. No.: 17/429,286

(22) PCT Filed: Feb. 12, 2020
(86) PCT No.: PCT/US20/17963

§ 371 Date: Aug. 6, 2021

Related U.S. Application Data
(60) Provisional application No. 62/805,097, filed on Feb. 13, 2019.

(51) Int. Cl. A61M 21/02

(52) U.S. Cl. CPC A61M 21/02; A61M 2021/005

(57) ABSTRACT

Disclosed herein are systems for use with a meditation session, said system comprising: a virtual reality display configured to display a virtual environment comprising a plurality of virtual images to a user while the user meditates; a biometric sensor configured to sense a plurality of biometric parameters of said user; and a processor configured to correlate said virtual environment or at least one virtual image of said plurality of virtual images with at least one biometric parameter of said plurality of biometric parameters.

### CROSS REFERENCE

This application claims the benefit of U.S. Provisional Application No. 62/805,097 filed Feb. 13, 2019, which is incorporated by reference herein in its entirety.

### BACKGROUND

Meditation has been shown to provide both physical and mental benefits to the human body which includes but is not limited to alleviated stress, reduced occurrence of anxiety attacks, improved mood and behavior, decreased tension related-pain, normalized blood pressure, improved immune system, etc. Millions of people use meditation for a variety of health-related purposes, among them, a lot are practicing meditation on a regular basis.

### SUMMARY OF THE INVENTION

Disclosed herein are methods, systems, and software for use in providing a meditation session to an individual. In some embodiments, the methods, systems, and software herein advantageous provide meditation sessions in which virtual reality (VR) experience, augmented reality (AR) experience, or otherwise virtual environment-related experience is utilized as an enhancement during at least part of a meditation session. In some embodiments, the systems, methods, and software herein enable quantitative feedback or evaluation of meditation. In some embodiments, such quantitative feedback or evaluation can be provided conveniently and efficiently before, during, and/or after a meditation session. In some embodiments, such quantitative feedback or evaluation can be provided conveniently and efficiently before, during, and/or after the VR and/or AR experience of the meditation session(s). In some embodiments, the methods, systems, and software herein can be used to allow modification or improvement based on the quantitative feedback(s) thereby significantly elevating the physical and/or mental benefits that traditional meditation can provide to individuals. In some embodiments, the methods, systems, and software described herein can be used to track progress of a mediation session including a therapeutic treatment, a health benefit, one or more metrics of an individual's health or mindset, or any combination thereof. In some embodiments, the methods, systems, and software described herein can be used to develop specific routines or specific recipes for the mediation session, each routine or recipe for the mediation session may target a different outcome, such as stress relief, reduction of blood pressure, pain relief or others. One or more parameters of a mediation session (such as a duration of a mediation session, a music selection, a virtual image selection, a brightness of the image, a humidity level of the room that the user is in, or any combination thereof, or others) may be modified to an ongoing mediation session or a future mediation session or both based on one or more biometric parameters measured. One or more parameters may be sensed continuously or intermittently, such as before a mediation session, during a mediation session, after a mediation session, or a combination thereof. A measured value of a parameter may be recorded. The measured value may be recorded continuously. A measured value of a parameter may be measured intermittently. A measured value of a parameter may be stored in a database.

Modifying a parameter of a mediation session may include modifying a duration of the mediation session, a presence or absence of a virtual image displayed, a presence or an absence of a sound projected, an intensity of a sound projected or an image displayed, a temperature level, a humidity level, or an ambient sound level of a room the user is in during the mediation session, a position of the user (standing, seating, lying down, or other), or any combination thereof. Modifying a parameter may results from a user input or a professional input. Modifying a parameter may result from a feedback of a biometric parameter measured by a sensor and provided to a controller of the system in which the controller may modified the parameter.

The systems and methods herein can advantageously facilitate improvement of a person's well-being through the use of a digital virtual environment, quantification of meditation or relaxation using biometric parameters, and correlation therebetween to optimize the effect of meditation. The systems and methods herein advantageously enable meditation-focused virtual experience (e.g., VR, augmented reality AR) that can show users how they can reach a demonstrable goal of relaxation. Using biometric parameters (e.g., heart rate, heart rate variability, breathing rate, pupil dilation, body posture, body temperature, oxygen level, blood pressure, moisture level of a skin surface, or others) as the data point and virtual environment as the vehicle for immersion, a user may see effect of medication and virtual environment on their biometrics in a short period of time, e.g., in 5 minutes. In some embodiments, the systems and methods include gathering insight from the user that can be acted upon throughout one or more meditation sessions, for example, color, scent, and sound. The systems and methods herein use data (e.g., from the user or other users) to drive positive change in their meditation, thereby providing significant improvement in the efficiency and effectiveness of traditional meditations.

In one aspect, disclosed herein is a system for use with a meditation session, said system comprising: a virtual reality display configured to display a virtual environment comprising a plurality of virtual images to a user while the user meditates; a biometric sensor configured to sense a plurality of biometric parameters of said user; and a processor configured to correlate said virtual environment or at least one virtual image of said plurality of virtual images with at least one biometric parameter of said plurality of biometric parameters. In some embodiments, said display is a head mounted display. In some embodiments, each of the plurality of virtual images comprises a portion of said virtual environment. In some embodiments, the virtual environment comprises a scene from nature. In some embodiments, the biometric sensor comprises a heart rate sensor, a blood pressure sensor, or an spO₂ sensor. In some embodiments, the biometric sensor is used to determine at least one of a heart rate variability or a respiratory rate. In some embodiments, the system further comprises: (d) an audio output device configured to provide a plurality of audio outputs to the user. In some embodiments, the at least one of the plurality of audio outputs corresponds to at least one of the plurality of virtual images. In some embodiments, the processor is further configured to correlate at least one of said audio outputs with at least one of said plurality of biometric parameters. In some embodiments, the processor is further configured to cause said audio output device to again output said at least one of said audio outputs during said meditation session or a subsequent meditation session. In some embodiments, the processor is further configured to cause said virtual reality display to display said at least one virtual image to said user during said meditation session or a subsequent meditation session. In some embodiments, the processor is further configured to cause said virtual reality display to again display said at least one virtual image to said user when said at least one biometric parameter is a certain value. In some embodiments, said biometric sensor comprises a heart rate sensor and said certain value is a heart rate less than about 70 beats per minute (bpm). In some embodiments, said biometric sensor comprises a heart rate sensor and said certain value is a systolic blood pressure less than about 130 mmHg. In some embodiments, the processor is further configured to cause said virtual reality display to again display said at least one virtual image to said user during said meditation session or a subsequent meditation session when said at least one biometric parameter is less than a baseline biometric parameter of the user that is previously sensed.

In another aspect, disclosed herein is a method for use during a meditation session, said method comprising: displaying a virtual environment comprising a plurality of virtual images to a user while the user meditates; sensing a plurality of biometric parameters of said user; and correlating, with a processor, said virtual environment or at least one virtual image of said plurality of virtual images with at least one biometric parameter of said plurality of biometric parameters. In some embodiments, said display is a head mounted display. In some embodiments, each of the plurality of virtual images comprises a portion of said virtual environment. In some embodiments, the virtual environment comprises a scene from nature. In some embodiments, the biometric sensor comprises a heart rate sensor, a blood pressure sensor, or an spO₂ sensor. In some embodiments, the biometric sensor is used to determine at least one of a heart rate variability or a respiratory rate. In some embodiments, the method further comprises: (d) outputting a plurality of audio outputs with an audio output device. In some embodiments, the at least one of the plurality of audio outputs corresponds to at least one of the plurality of virtual images. In some embodiments, the processor is further configured to correlate at least one of said audio outputs with at least one of said plurality of biometric parameters. In some embodiments, the processor is further configured to cause said audio output device to again output said at least one of said audio outputs during said meditation session or a subsequent meditation session. In some embodiments, the processor is further configured to cause said virtual reality display to display said at least one virtual image to said user during said meditation session or a subsequent meditation session. In some embodiments, the processor is further configured to cause said virtual reality display to again display said at least one virtual image to said user when said at least one biometric parameter is a certain value. In some embodiments, said biometric sensor comprises a heart rate sensor and said certain value is a heart rate less than about 70 beats per minute (bpm). In some embodiments, said biometric sensor comprises a heart rate sensor and said certain value is a systolic blood pressure less than about 130 mmHg. In some embodiments, the processor is further configured to cause said virtual reality display to again display said at least one virtual image to said user during said meditation session or a subsequent meditation session when said at least one biometric parameter is less than a baseline biometric parameter of the user that is previously sensed.

In yet another aspect, disclosed herein is a non-transitory computer readable storage medium comprising computer readable software configured to cause a processor to: display a virtual environment comprising a plurality of virtual images to a user while the user meditates; sense a plurality of biometric parameters of said user; and correlate said virtual environment or at least one virtual image of said plurality of virtual images with at least one biometric parameter of said plurality of biometric parameters. In some embodiments, said display is a head mounted display. In some embodiments, each of the plurality of virtual images comprises a portion of said virtual environment. In some embodiments, the virtual environment comprises a scene from nature. In some embodiments, the biometric sensor comprises a heart rate sensor, a blood pressure sensor, or an spO₂ sensor. In some embodiments, the biometric sensor is used to determine at least one of a heart rate variability or a respiratory rate. In some embodiments, the software is further configured to cause the processor to: (d) output a plurality of audio outputs to the user. In some embodiments, the at least one of the plurality of audio outputs corresponds to at least one of the plurality of virtual images. In some embodiments, the processor is further configured to correlate at least one of said audio outputs with at least one of said plurality of biometric parameters. In some embodiments, the processor is further configured to cause said audio output device to again output said at least one of said audio outputs during said meditation session or a subsequent meditation session. In some embodiments, the processor is further configured to cause said virtual reality display to display said at least one virtual image to said user during said meditation session or a subsequent meditation session. In some embodiments, the processor is further configured to cause said virtual reality display to again display said at least one virtual image to said user when said at least one biometric parameter is a certain value. In some embodiments, said biometric sensor comprises a heart rate sensor and said certain value is a heart rate less than about 70 beats per minutes (bpm). In some embodiments, wherein said biometric sensor comprises a heart rate sensor and said certain value is a systolic blood pressure less than about 130 mmHg. In some embodiments, the processor is further configured to cause said virtual reality display to again display said at least one virtual image to said user during said meditation session or a subsequent meditation session when said at least one biometric parameter is less than a baseline biometric parameter of the user that is previously sensed.

In one aspect, disclosed herein is a system for use with a meditation session, said system comprising: a virtual reality display configured to display a virtual environment comprising a plurality of virtual images to a user while the user meditates; a biometric sensor configured to sense a plurality of biometric parameters of said user; and a processor configured to correlate said virtual environment or at least one virtual image of said plurality of virtual images with at least one biometric parameter of said plurality of biometric parameters. In some embodiments, said display is a head mounted display. In some embodiments, each of the plurality of virtual images comprises a portion of said virtual environment. In some embodiments, the virtual environment comprises a scene from nature. In some embodiments, the biometric sensor comprises a heart rate sensor, a blood pressure sensor, or an spO₂ sensor. In some embodiments, the biometric sensor is used to determine at least one of a heart rate variability or a respiratory rate. In some embodiments, the system further comprises: (d) an audio output device configured to provide a plurality of audio outputs to the user. In some embodiments, the at least one of the plurality of audio outputs corresponds to at least one of the plurality of virtual images. In some embodiments, the processor is further configured to correlate at least one of said audio outputs with at least one of said plurality of biometric parameters. In some embodiments, the processor is further configured to cause said audio output device to again output said at least one of said audio outputs during said meditation session or a subsequent meditation session. In some embodiments, the processor is further configured to cause said virtual reality display to display said at least one virtual image to said user during said meditation session or a subsequent meditation session. In some embodiments, the processor is further configured to cause said virtual reality display to again display said at least one virtual image to said user when said at least one biometric parameter is a certain value. In some embodiments, said biometric sensor comprises a heart rate sensor and said certain value is a heart rate less than about 70 beats per minute (bpm). In some embodiments, said biometric sensor comprises a heart rate sensor and said certain value is a systolic blood pressure less than about 130 mmHg. In some embodiments, the processor is further configured to cause said virtual reality display to again display said at least one virtual image to said user during said meditation session or a subsequent meditation session when said at least one biometric parameter is less than a baseline biometric parameter of the user that is previously sensed.

In another aspect, disclosed herein is a method for use during a meditation session, said method comprising: displaying a virtual environment comprising a plurality of virtual images to a user while the user meditates; sensing a plurality of biometric parameters of said user; and correlating, with a processor, said virtual environment or at least one virtual image of said plurality of virtual images with at least one biometric parameter of said plurality of biometric parameters. In some embodiments, said display is a head mounted display. In some embodiments, each of the plurality of virtual images comprises a portion of said virtual environment. In some embodiments, the virtual environment comprises a scene from nature. In some embodiments, the biometric sensor comprises a heart rate sensor, a blood pressure sensor, or an spO₂ sensor. In some embodiments, the biometric sensor is used to determine at least one of a heart rate variability or a respiratory rate. In some embodiments, the method further comprises: (d) outputting a plurality of audio outputs with an audio output device. In some embodiments, the at least one of the plurality of audio outputs corresponds to at least one of the plurality of virtual images. In some embodiments, the processor is further configured to correlate at least one of said audio outputs with at least one of said plurality of biometric parameters. In some embodiments, the processor is further configured to cause said audio output device to again output said at least one of said audio outputs during said meditation session or a subsequent meditation session. In some embodiments, the processor is further configured to cause said virtual reality display to display said at least one virtual image to said user during said meditation session or a subsequent meditation session. In some embodiments, the processor is further configured to cause said virtual reality display to again display said at least one virtual image to said user when said at least one biometric parameter is a certain value. In some embodiments, said biometric sensor comprises a heart rate sensor and said certain value is a heart rate less than about 70 beats per minute (bpm). In some embodiments, said biometric sensor comprises a heart rate sensor and said certain value is a systolic blood pressure less than about 130 mmHg. In some embodiments, the processor is further configured to cause said virtual reality display to again display said at least one virtual image to said user during said meditation session or a subsequent meditation session when said at least one biometric parameter is less than a baseline biometric parameter of the user that is previously sensed.

In yet another aspect, disclosed herein is a non-transitory computer readable storage medium comprising computer readable software configured to cause a processor to: display a virtual environment comprising a plurality of virtual images to a user while the user meditates; sense a plurality of biometric parameters of said user; and correlate said virtual environment or at least one virtual image of said plurality of virtual images with at least one biometric parameter of said plurality of biometric parameters. In some embodiments, said display is a head mounted display. In some embodiments, each of the plurality of virtual images comprises a portion of said virtual environment. In some embodiments, the virtual environment comprises a scene from nature. In some embodiments, the biometric sensor comprises a heart rate sensor, a blood pressure sensor, or an spO₂ sensor. In some embodiments, the biometric sensor is used to determine at least one of a heart rate variability or a respiratory rate. In some embodiments, the software is further configured to cause the processor to: (d) output a plurality of audio outputs to the user. In some embodiments, the at least one of the plurality of audio outputs corresponds to at least one of the plurality of virtual images. In some embodiments, the processor is further configured to correlate at least one of said audio outputs with at least one of said plurality of biometric parameters. In some embodiments, the processor is further configured to cause said audio output device to again output said at least one of said audio outputs during said meditation session or a subsequent meditation session. In some embodiments, the processor is further configured to cause said virtual reality display to display said at least one virtual image to said user during said meditation session or a subsequent meditation session. In some embodiments, the processor is further configured to cause said virtual reality display to again display said at least one virtual image to said user when said at least one biometric parameter is a certain value. In some embodiments, said biometric sensor comprises a heart rate sensor and said certain value is a heart rate less than about 70 beats per minute (bpm). In some embodiments, wherein said biometric sensor comprises a heart rate sensor and said certain value is a systolic blood pressure less than about 130 mmHg. In some embodiments, the processor is further configured to cause said virtual reality display to again display said at least one virtual image to said user during said meditation session or a subsequent meditation session when said at least one biometric parameter is less than a baseline biometric parameter of the user that is previously sensed.

Also disclosed herein is a computer implemented method for providing a meditation session, said method comprising: sensing at least one parameter from an individual while said individual is meditating during a meditation session; inputting the at least one parameter into a machine learning software module; and determining, with the machine learning software module, a modification of the meditation session. In some embodiments, the method comprises displaying, with a display, a virtual environment comprising a plurality of virtual images to a user while the user meditates. In some embodiments, said display is a head mounted display. In some embodiments, each of the plurality of virtual images comprises a portion of said virtual or augmented environment. In some embodiments, the virtual environment comprises a scene from nature. In some embodiments, the at least one parameter comprises a heart rate, a blood pressure, or an spO₂. In some embodiments, the at least one parameter comprises at least one of a heart rate variability or a respiratory rate. In some embodiments, outputting a plurality of audio outputs to the individual. In some embodiments, the at least one of the plurality of audio outputs corresponds to at least one of a plurality of virtual images. In some embodiments, the method comprises correlating at least one of said audio outputs with the at least one parameter. In some embodiments, the processor is further configured to cause said audio output device to again output said at least one of said audio outputs during said meditation session or a subsequent meditation session.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1 shows a non-limiting exemplary schematic diagram of the system for providing a VR-enhanced quantitative meditation session to a user;

FIG. 2 shows a non-limiting exemplary process flow of the method for providing a quantitative meditation session to an individual;

FIG. 3 shows a non-limiting schematic diagram of a digital processing device; in this case, a device with one or more CPUs, a memory, a communication interface, and a display;

FIG. 4 shows a non-limiting schematic diagram of a web/mobile application provision system; in this case, a system providing browser-based and/or native mobile user interfaces; and

FIG. 5 shows a non-limiting schematic diagram of a cloud-based web/mobile application provision system; in this case, a system comprising an elastically load balanced, auto-scaling web server and application server resources as well synchronously replicated databases.

### DETAILED DESCRIPTION OF THE INVENTION

Disclosed herein, in certain embodiments, is a system for use with a meditation session, said system comprising: a virtual reality display configured to display a virtual environment comprising a plurality of virtual images to a user while the user meditates; a biometric sensor configured to sense a plurality of biometric parameters of said user; and a processor configured to correlate said virtual environment or at least one virtual image of said plurality of virtual images with at least one biometric parameter of said plurality of biometric parameters. In some embodiments, said display is a head mounted display. In some embodiments, each of the plurality of virtual images comprises a portion of said virtual environment. In some embodiments, the virtual environment comprises a scene from nature. In some embodiments, the biometric sensor comprises a heart rate sensor, a blood pressure sensor, or an spO₂ sensor. In some embodiments, the biometric sensor is used to determine at least one of a heart rate variability or a respiratory rate. In some embodiments, the system further comprises: (d) an audio output device configured to provide a plurality of audio outputs to the user. In some embodiments, the at least one of the plurality of audio outputs corresponds to at least one of the plurality of virtual images. In some embodiments, the processor is further configured to correlate at least one of said audio outputs with at least one of said plurality of biometric parameters. In some embodiments, the processor is further configured to cause said audio output device to again output said at least one of said audio outputs during said meditation session or a subsequent meditation session. In some embodiments, the processor is further configured to cause said virtual reality display to display said at least one virtual image to said user during said meditation session or a subsequent meditation session. In some embodiments, the processor is further configured to cause said virtual reality display to again display said at least one virtual image to said user when said at least one biometric parameter is a certain value. In some embodiments, said biometric sensor comprises a heart rate sensor and said certain value is a heart rate less than about 70 beats per minute (bpm). In some embodiments, said biometric sensor comprises a heart rate sensor and said certain value is a systolic blood pressure less than about 130 mmHg. In some embodiments, the processor is further configured to cause said virtual reality display to again display said at least one virtual image to said user during said meditation session or a subsequent meditation session when said at least one biometric parameter is less than a baseline biometric parameter of the user that is previously sensed.

Disclosed herein, in certain embodiments, is a method for use during a meditation session, said method comprising: displaying a virtual environment comprising a plurality of virtual images to a user while the user meditates; sensing a plurality of biometric parameters of said user; and correlating, with a processor, said virtual environment or at least one virtual image of said plurality of virtual images with at least one biometric parameter of said plurality of biometric parameters. In some embodiments, said display is a head mounted display. In some embodiments, each of the plurality of virtual images comprises a portion of said virtual environment. In some embodiments, the virtual environment comprises a scene from nature. In some embodiments, the biometric sensor comprises a heart rate sensor, a blood pressure sensor, or an spO₂ sensor. In some embodiments, the biometric sensor is used to determine at least one of a heart rate variability or a respiratory rate. In some embodiments, the method further comprises: (d) outputting a plurality of audio outputs with an audio output device. In some embodiments, the at least one of the plurality of audio outputs corresponds to at least one of the plurality of virtual images. In some embodiments, the processor is further configured to correlate at least one of said audio outputs with at least one of said plurality of biometric parameters. In some embodiments, the processor is further configured to cause said audio output device to again output said at least one of said audio outputs during said meditation session or a subsequent meditation session. In some embodiments, the processor is further configured to cause said virtual reality display to display said at least one virtual image to said user during said meditation session or a subsequent meditation session. In some embodiments, the processor is further configured to cause said virtual reality display to again display said at least one virtual image to said user when said at least one biometric parameter is a certain value. In some embodiments, said biometric sensor comprises a heart rate sensor and said certain value is a heart rate less than about 70 beats per minute (bpm). In some embodiments, said biometric sensor comprises a heart rate sensor and said certain value is a systolic blood pressure less than about 130 mmHg. In some embodiments, the processor is further configured to cause said virtual reality display to again display said at least one virtual image to said user during said meditation session or a subsequent meditation session when said at least one biometric parameter is less than a baseline biometric parameter of the user that is previously sensed.

Disclosed herein, in certain embodiments, is a non-transitory computer readable storage medium comprising computer readable software configured to cause a processor to: display a virtual environment comprising a plurality of virtual images to a user while the user meditates; sense a plurality of biometric parameters of said user; and correlate said virtual environment or at least one virtual image of said plurality of virtual images with at least one biometric parameter of said plurality of biometric parameters. In some embodiments, said display is a head mounted display. In some embodiments, each of the plurality of virtual images comprises a portion of said virtual environment. In some embodiments, the virtual environment comprises a scene from nature. In some embodiments, the biometric sensor comprises a heart rate sensor, a blood pressure sensor, or an spO₂ sensor. In some embodiments, the biometric sensor is used to determine at least one of a heart rate variability or a respiratory rate. In some embodiments, the software is further configured to cause the processor to: (d) output a plurality of audio outputs to the user. In some embodiments, the at least one of the plurality of audio outputs corresponds to at least one of the plurality of virtual images. In some embodiments, the processor is further configured to correlate at least one of said audio outputs with at least one of said plurality of biometric parameters. In some embodiments, the processor is further configured to cause said audio output device to again output said at least one of said audio outputs during said meditation session or a subsequent meditation session. In some embodiments, the processor is further configured to cause said virtual reality display to display said at least one virtual image to said user during said meditation session or a subsequent meditation session. In some embodiments, the processor is further configured to cause said virtual reality display to again display said at least one virtual image to said user when said at least one biometric parameter is a certain value. In some embodiments, said biometric sensor comprises a heart rate sensor and said certain value is a heart rate less than about 70 bpm. In some embodiments, wherein said biometric sensor comprises a heart rate sensor and said certain value is a systolic blood pressure less than about 130 mmHg. In some embodiments, the processor is further configured to cause said virtual reality display to again display said at least one virtual image to said user during said meditation session or a subsequent meditation session when said at least one biometric parameter is less than a baseline biometric parameter of the user that is previously sensed.

### Certain Terms

Unless otherwise defined, all technical terms used herein have the same meaning as commonly understood by one of ordinary skill in the art to which this invention belongs.

As used herein, the singular forms “a,” “an,” and “the” include plural references unless the context clearly dictates otherwise. Any reference to “or” herein is intended to encompass “and/or” unless otherwise stated.

As used herein, the term “about” refers to an amount that is near the stated amount by about 10%, 5%, or 1%, including increments therein.

As used herein, the term “meditation” is equivalent to a meditation session which can include different portions. In some embodiments, the meditation herein includes a time duration, e.g., about: 5 minutes, 10 minutes, 15 minutes, 20 minutes, 25 minutes, 30 minutes, 35 minutes, 40 minutes, 45 minutes, 50 minutes, 55 minutes, 60 minutes, 1.25 hours, 1.5 hours, 1.75 hours, 2 hours, or any other time duration. In some embodiments, a time duration of a mediation session may be from about 5 minutes to about 60 minutes. In some embodiments, a time duration of a mediation session may be from about 5 minutes to about 2 hours. In some embodiments, a time duration of a mediation session may be from about 5 minutes to about 30 minutes. In some embodiments a time duration of a mediation session may be from about 5 minutes to about 20 minutes. In some embodiments a time duration of a mediation session may be at least about: 5 minutes, 10 minutes, 15 minutes, 20 minutes, 25 minutes, 30 minutes, 35 minutes, 40 minutes, 45 minutes, 50 minutes, 55 minutes, 60 minutes, 1.25 hours, 1.5 hours, 1.75 hours, 2 hours, or more. In some embodiments, a portion of a meditation session can be from about 1% to about 99% of the session.

As used herein, the term “virtual environment” can include augmented reality (“AR”), Virtual reality (“VR”) technology, or any other technology that may display virtual and/or real environment to the user. Herein, “augmented reality” or “AR” might refer to virtual overlay of simulated constructs either over actual views of actual objects and settings or over images of actual objects and settings, while “virtual reality” or “VR” might refer to an enclosed sensory environment where everything that is observed by the user is simulated, and “mixed reality” or “M×R” might refer to a combination of AR and VR (e.g., a VR presentation in which AR elements are simulated are embedded in the presentation, or the like).

As used herein the AR technology can provide a live direct or indirect view of a physical, real-world environment whose elements are augmented (or supplemented) by computer-generated sensory input such as graphics, video, sound, or GPS data.

As used herein, the VR technology, on the other hand, can utilize software to generate realistic images, sounds and other sensations that replicate a real environment (or create an imaginary setting), and simulate a user's physical presence in this environment, by enabling the user to interact with this space and any objects depicted therein using specialized display screens or projectors and other devices.

As used herein, a user herein can be any individual using the systems and methods herein, and/or a user that is a recipient of the meditation session(s). A user herein can be equivalent to an individual.

Systems for Quantitative Meditation with Enhancement

In some embodiments, disclosed herein are quantitative meditations that are enhanced with VR, AR, M×R, or other virtual environment-related technologies. In some embodiments, disclosed herein are quantitative meditations that use sensing of parameter(s) of a human subject or a user to guide meditation and/or display to the user using VR, AR, or other technologies based on the parameter(s).

In some embodiments, the parameters of the subject or user can include any biological or physiological parameters. In some embodiments, the parameters of the subject or user can include any biometric parameters of the user. A biometric parameter may include one or more of the following: a brain wave, a level of brain activity, a level of brain activity in a portion of a brain region, a blood pressure, a heart rate, a pupil dilation, a body posture, a body temperature, an oxygen level, or a moisture level of a skin surface). In some embodiments, the parameters can be sensed using one or more sensors.

In some embodiments, a system may comprise about: 2, 3, 4, 5, 6, 7, 8, 9, 10 sensors or more. In some embodiments, a system may comprise from about 1 sensors to about 10 sensors. In some embodiments, a system may comprise from about 1 sensor to about 5 sensors. In some embodiments, a system may comprise from about 1 sensor to about 3 sensors.

In some embodiments, about: 2, 3, 4, 5, 6, 7, 8, 9, 10 sensors or more may be employed during a mediation session to measure one or more parameters. In some embodiments, from about 1 sensors to about 10 sensors may be employed. In some embodiments, from about 1 sensor to about 5 sensors may be employed. In some embodiments, from about 1 sensor to about 3 sensors may be employed.

A sensor may contact a surface of an individual's body. A sensor may be proximal a surface of an individual's body. A sensor may be attached to an individual's body. A sensor may be configured as part of a system as described herein, such as configured as part of a virtual reality headset (such as a sensor for sensing a brain wave or brain activity or pupil dilation), or configured as part of a keyboard or remote that a user may hold (such as a sensor for heart rate or skin moisture level) or configured as part of a chair that a user sits in during the mediation session, or configured as part of an earbud that the user may insert in an ear, or any combination thereof. A sensor may be operated by the individual. A sensor may be operated by a professional, such as a mediation provider. A sensor may be as part of a recipe for a mediation session, wherein a controller instructs operation of the sensor. In some embodiments, the systems and methods herein include software or computer programs that can evaluate the values of the sensed parameter(s) and plan out schemes of future display to the user and/or guidance to the user regarding meditation. In some embodiments, the values of the sensed parameter(s) can be evaluated automatically, and future displays can be designed automatically using the parameters. As such, the effectiveness, efficiency, and/or quality of the meditation can be efficiently and reliably improved.

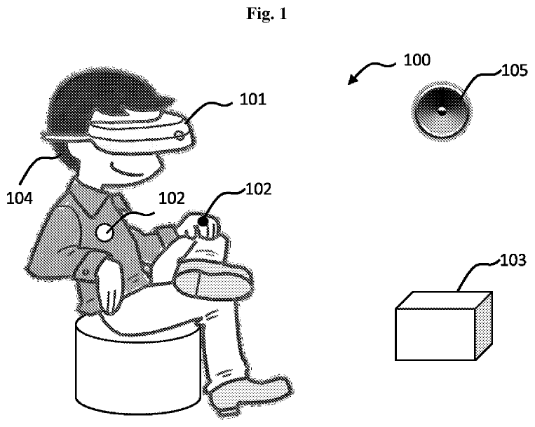

As shown in FIG. 1, in some embodiments, disclosed herein is a system 100 for use with a meditation session. In some embodiments, the system 100 comprises: a digital display 101 configured to display a virtual environment comprising a plurality of virtual images to a user 104 while the user meditates. The system can include one or more sensors (e.g., biometric sensors) 102 configured to sense a plurality of parameters of said user (e.g., biometric parameters); and a processor or a digital processing device 103 configured to correlate said virtual environment or at least one virtual image of said plurality of virtual images with at least one parameter of said plurality of parameters.

In some embodiments, the digital display 101 is head-mounted. In some embodiments, the digital display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the digital processing device, such as a VR headset or AR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In some embodiments, the digital display includes a head mountable device. In some embodiments, the digital display includes a look gaze based system for selecting the meditation environment.

In some embodiments, the virtual environment herein is a VR environment. In some embodiments, the virtual environment is an AR environment. In some embodiments, the virtual environment herein is a M×R environment. In some embodiments, the virtual environment comprises a scene from nature. In some embodiments, the virtual environment comprises a scene that is not in the actual environment that the user is in. In some embodiments, the virtual environment comprises one or more sensational effect selected from but not limited to: visual, audio, olfactory, temperature, tactile, and balance. In some embodiments, each of the plurality of virtual images comprises a portion of the virtual environment. In some embodiments, the virtual environment does not include any element that is in the actual environment of the user or a virtual representation of any element of the actual environment of the user. In some embodiments, the virtual environment does not include a virtual representation of the user. In some embodiments, the virtual environment includes a virtual representation of the user, e.g., an avatar or an image of the user.

In some embodiments, the sensor(s) 102 herein includes one or more biometric sensors. In some embodiments, the biometric sensor comprises a heart rate sensor, a blood pressure sensor, or an spO₂ sensor. In some embodiments, the biometric sensor is used to determine at least one of a heart rate variability or a respiratory rate.

In some embodiments, the sensors herein are configured for sensing one or more parameters of the user and result in one or more numerical values with or without unit. In some embodiments, sensing of the parameter(s) may result in multiple values that can be numerical, e.g., sensing result can be an image of a certain portion of the user. In some embodiments, one sensor can generate one or multiple sensed valued for one or more parameters.

In some embodiments, the sensed parameters are used alone in order to generate adjustment of the virtual environment. In some embodiments, the sensed parameters are combined with the patient or the user's input to generate the adjustment of the virtual environment. In some embodiments, the sensed parameters are combined with other information of the user to generate the adjustment of the virtual environment. As a non-limiting example, such other information can include demographic information of the user. As yet another example, such other information can include historical biometric data of the user in previous meditation sessions. As yet another non-limiting example, the sensed elevation of the user's heart rate may be used to provide soothing nature images with the user's favorite music theme in the virtual environment to be presented to the user.

In some embodiments, the parameter comprises a temperature of a portion of a body of the user. The sensor may comprise a thermographic camera, a temperature probe, and/or pad. In some embodiments, the parameter comprises a vital sign of the user. In some embodiments, the parameter(s) include an electrocardiogram (ECG) of the user. The sensor may comprise at least one ECG electrode. In some embodiments, the parameter(s) comprises an electroencephalogram (EEG), and the sensor comprises at least one EEG sensor.

In some embodiments, the sensed parameters are data having time stamps that correspond to events within the experience so that it can be easy to correlate biometric feedback to what the user is experiencing. The meditation session can include one or more of the following that can be correlated with the user's sensed parameters: environment previews, major events during intro; environmental selection; start and end of meditation session; and key events during outro.

In some embodiments, the virtual environment, the audio output, or at least a portion of a meditation session can be savable and exportable to a pre-determined format compatible with one or more software or applications.

In some embodiments, the sensor, e.g., one or more cameras, can be mounted overhead on the ceiling or on any fixed structural element above the user. In some embodiments, the sensor is attached to a movable element, for example a movable arm which is mounted to a table or a transportable cart.

In some embodiments, each sensor herein includes one or more markers or indicators that facilitate indication or identification of the sensor(s) relative to the user's position. In some embodiments, the markers or indicators can help a user to locate the sensor(s) relative to the individual. In some embodiments, the markers or indicators can be visualized or otherwise identified in a mobile application or web application so that the user can locate the markers thus the sensors relative to the individual. In some embodiments, such markers or indicators can advantageously facilitate positioning of the user, for example, in a consistent place in relation to the sensor(s), e.g., camera, heart rate sensor, respiration sensor, etc. In some embodiments, such markers or indicators may advantageously minimize bias that may be caused by inconsistent positioning of the user relative to the sensor(s).

In some embodiments, the parameter includes one or more of a respiration rate, oxygenation, heart rate, heart rhythm, blood pressure, blood glucose level, muscle action potential, and brain function. In some embodiments, the parameter includes a thermal reading.

In some embodiments, the sensor is placed on at least a portion of the body of the individual. For example, an EEG sensor or one or more EEG leads are attached to the chest of the individual. As another example, a blood oxygen sensor can be clipped on a finger of the individual. In some embodiments, the sensor is in contact with at least a portion of the body of the individual. As non-limiting examples, the sensor can be placed on a piece of clothing, or any other objects that the user may contact. In some embodiments, the sensor is not in direct contact with the user, e.g., a camera. In some embodiments, the sensor herein includes but is not limited to one or more of: a temperature sensor, a humidity sensor, an electrical impedance sensor, an acoustic impedance sensor, an electromyography (EMG) sensor, an oxygen sensor, a pH sensor, an optical sensor, an ultrasound sensor, a glucose sensor, a biomarker sensor, a heart rate monitor, a respirometer, an electrolyte sensor, a blood pressure sensor, an EEG sensor, an ECG sensor, a body hydration sensor, a carbon dioxide sensor, a carbon monoxide sensor, a blood alcohol sensor, and a Geiger counter.

In some embodiments, the sensor herein is set-up so that it may minimize the discomfort it may cause the user. In some embodiments, the sensor herein is set-up so that the interference to the user's privacy is minimized. In some embodiments, the user may be provided with options as to how the sensor is set-up. As an example, the user may not want any sensor to be attached to his body, and he can select the sensor that is embedded on a chair back and can contact his body while he sits in the chair. A sensor may be configured as part of a virtual reality headset. A sensor may be configured as part of an ear bud or finger clamp. A sensor may be configured as part of a strap or pad that may be attached to a surface of a user. A sensor may be configured as part of a user remote, user console, or user interface with which the user may interact. A sensor may be configured as part of a chair, a table, a bed, or surface that a user may stand, sit, lay, or rest upon. A sensor may be configured as part of a room that the user occupies during a mediation session.

In some embodiments, the methods, systems, and software herein utilize one or more sensed parameters to guide content of the virtual environment in a subsequent portion or sessions of a meditation session. In some embodiments, guiding content of the virtual environment in a subsequent portion or sessions of meditation includes modifying one or more virtual images, audio, temperature, tactile, or other output that can be controlled by the processor to be presented to the user. For example, changing a background music, changing a saturation of the virtual images, changing a brightness of the images, changing a humidity level in the room that the user is in, etc.

In some embodiments, the system 100 further comprises an audio output device 105 configured to provide a plurality of audio outputs to the user. In some embodiments, the plurality of audio outputs corresponds to or is related to at least one of the plurality of virtual images of the virtual environment.

In some embodiments, the audio output device includes one or more selected from but is not limited to: a speaker, an earphone, and a headset.

In some embodiments, the system 100 herein includes a processor 103. The processor can be in communication with one or more of the digital displays 101, the sensors 102, and the audio output device 105. Such communication can be wired or wireless communication. Such communication can be uni-directional or bi-directional so that data and/or command can be communicated therebeween.

In some embodiments, the processor 103 herein is configured to execute code or software stored on an electronic storage location of a digital processing device such as, for example, on the memory. In some embodiments, the processor herein includes a central processing unit (CPU).

In some embodiments, the processor 103 herein is configured to correlate at least a portion of the virtual environment (e.g., one or more of a virtual image, an audio output, a scent, a temperature, or a combination thereof) with one or more sensed parameters of the user. In some embodiments, such correlation can be used as a feedback to adjust display of the current virtual environment in a current meditation session or to plan a future virtual environment in a subsequent meditation session.

In some embodiments, the processor 103 is configured to correlate audio output(s) with at least one parameter that has been sensed. In some embodiments, the processor can cause the audio output device 105 to repeat outputting one or more audio outputs during a current meditation session or a subsequent meditation session. In some embodiments, the processor is configured to cause the virtual reality display 101 to repeat displaying of one or more virtual image to the user during said meditation session or a subsequent meditation session. In some embodiments, the processor is configured to cause said virtual reality display to repeat displaying of one or more virtual images to the user when one or more sensed parameters are of a certain pre-determined value or in a certain pre-determined range. For example, when the sensors 102 include a heart rate sensor and the processor can control the digital display or the audio output device to repeat output of image(s) or audio output(s) when the sensed heart rate is less than about 70 bpm. As another example, when the sensors 102 include a blood pressure sensor and the processor can control the digital display or the audio output device to repeat output of image(s) or audio output(s) when a systolic blood pressure is less than about 130 mmHg. In some embodiments, the processor is configured to cause the virtual reality display to again display at least one virtual image to the user during a current meditation session or a subsequent meditation session when one or more sensed parameter is different from a baseline parameter of the user that is previously sensed (e.g., less than or greater than) or out of a baseline parameter range of the user that is previously sensed. In some embodiments, the processor is configured to cause the audio output device to again output the audio output(s) to the user during a current meditation session or a subsequent meditation session when one or more sensed parameter is different from a baseline parameter of the user that is previously sensed (e.g., less than or greater than) or out of a baseline parameter range of the user that is previously sensed. In some embodiments, the system 100 is a computer-implemented system. In some embodiments, the system includes a digital processing device having one or more processors 103. In some embodiments, the system herein includes one or more computer program or algorithm. In some embodiments, the system herein includes a database. In some embodiments, the processor is configured to execute one or more computer program or algorithm herein to generate results that are associated with correlation between the virtual environment and the sensed parameter(s).

In some embodiments, the processor can control one or more other elements of the system herein, such as the digital display, the sensor, and the audio output device. In some embodiments, the processor controls to turn on/off of one or more elements of the system. In some embodiments, the processor controls to sense, transmit, or store the parameter(s). In some embodiments, the processor processes the parameter(s) to determine the adjustment to the current virtual environment or plan for future virtual environment. In some embodiments, the processor utilizes the machine learning algorithm to determine information related to the current or future virtual environment.

In some embodiments, the system includes a digital processing device that can control the digital display and/or the audio output device so that the virtual environment and/or audio outputs can be presented automatically, at least in part. In some embodiments, the digital processing device can control the elements of the systems disclosed herein with wire or wirelessly.

In some embodiments, the system includes a non-transitory computer readable medium configured to receive information regarding the virtual environment, the sensed parameter (s), and outputs correlation between the virtual environment and the parameters. In some embodiments, the correlation is used to modify, start, or cease a presentation of the virtual environment to the user (e.g., one or more virtual images).

In some embodiments, the system herein includes a remote server configured to receive and analyze the parameter, the signal, or any other data communicated to the remote server. In some embodiments, the remote server includes a digital processing device. In some embodiments, the remote server includes a database. In some embodiments, the remote server includes a computer program. In some embodiments, the remote server includes a user interface that allows a user to edit/view functions of the remote server. For example, the user interface allows a user to set a fixed interval, e.g., about 1 hour, 2 hours, 3 hours, 4 hours, 5 hours, 6 hours, 7 hours, 8 hours, 9 hours, 10 hours, 11 hours, 12 hours, or more for data to be communicated from the sensor(s) to the server to be saved.

### VR Enhanced Quantitative Meditation Methods

In some embodiments, disclosed herein are methods for use during a meditation session. In some embodiments, the method herein comprises displaying or otherwise presenting a virtual environment to a user while the user meditates using a digital display. The virtual environment may include a plurality of virtual images. In some embodiments, the method includes sensing a plurality of parameters of the user, e.g., biometric parameters. Such sensing can be before, during, or after displaying the virtual environment to the user. In some embodiments, such sensing can be before, during, or after a meditation session. In some embodiments, such sensing can be during at least a portion of displaying the virtual environment to the user and/or the meditation session. In some embodiments, the method includes correlating the virtual environment (e.g., at least one virtual image of said plurality of virtual images) with at least one parameter sensed using the sensor(s). In some embodiments, the method includes outputting a plurality of audio outputs with an audio output device to the user at least during a portion of a meditation session. In some embodiments, the at least one of the plurality of audio outputs corresponds to or is independent of at least one of the plurality of virtual images.

In some embodiments, the method includes correlating at least one of said audio outputs with at least one sensed parameter. In some embodiments, the correlation of the virtual environment with the parameter(s) and the correlation of the audio outputs with the sensed parameter(s) can be separated correlations or a combined correlation. In some embodiments, the method includes an operation of causing the audio output device to repeat outputting at least one of said audio outputs during said meditation session or a subsequent meditation session. In some embodiments, the method includes an operation of repeat displaying of at least one virtual image to the user during the meditation session or a subsequent meditation session. In some embodiments, the method includes causing the virtual reality display and/or the audio output device to repeat displaying virtual images or outputting audio outputs to the user when at least one biometric parameter is a certain value or within a certain range. In some embodiments, the certain range can be determined by the user, a medical professional, or automatically by a computer program. In some embodiments, the certain value is a heart rate less than about 70 bpm or greater than about 90 bpm. In some embodiments, the certain value is a heart rate less than about 60 bpm, 70 bpm, about 80 bpm, or about 90 bpm, or greater than about 70 bpm, about 80 bpm, about 90 bpm, or about 100 bpm. In some embodiments, the certain value is a systolic blood pressure less than about 100 mm Hg, about 110 mmHg, about 120 mmHg, or about 130 mmHg, or greater than about 160 mmHg, about 170 mmHg, about 180 mmHg, about 190 mmHg, or about 200 mmHg.

In some embodiments, the method includes causing the virtual reality display to repeat displaying virtual images and/or the audio output device to repeat outputting audio outputs to the user during a current meditation session or a subsequent meditation session when at least one biometric parameter is less than or greater than a baseline biometric parameter of the user that is previously sensed.

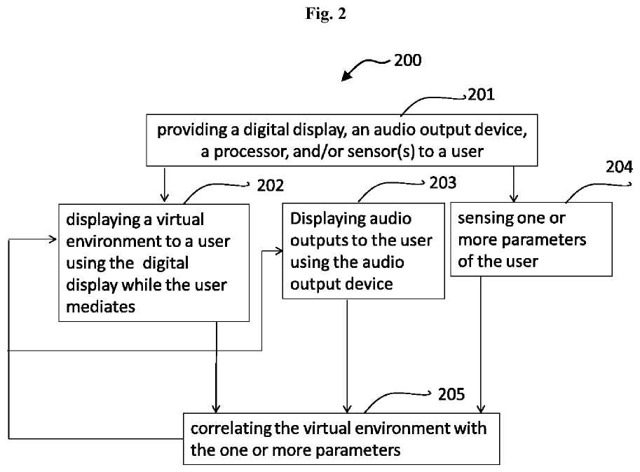

Referring to FIG. 2, in a particular embodiment, the method 200 for providing a quantitative meditation to an individual 104 may include an operation that provides a digital display, an audio output device, a processor, and/or one or more sensors to the user 201 before a meditation session starts. In the same embodiment, subsequent to operation 201, the method optionally includes instructing the user or positioning the user relative to the digital display, the audio output device, and/or the sensor(s) for preparation of the meditation session. Subsequently, in the same embodiment, the method includes displaying a virtual environment to the user using the digital display 202 at least during a portion of the meditation session. In this particular embodiment, the method may include presenting other sensory effects such as audio outputs using the audio output device to the user 203, either simultaneously and correspondingly with the operation 202 or independent of operation 202. In some embodiments, sensory effects other than visual and/or audio effects can be presented to the user either correspondingly with operations 202 and/or 203 or independently. In the same embodiments, the sensor(s) are sensing one or more parameters 204 of the user at least during a portion of the operations 202 and/or 203. Subsequently, the method herein can correlate the virtual environment, the audio output and/or other sensory effect with the one or more sensed parameters 205. Such correlation in operation 205 can be used to guide current or future meditation sessions, more specifically, future operations 202 and/or 203 for at least a portion of a current meditation session or future meditation sessions. In this embodiment, operation 205 enables a quantitative feedback to the meditation session that improves the effectiveness of the meditation. In some embodiments, the method can stop without performing operation 205. In some embodiments, (the sequence of i) operations 202 203, 204; and 2) operation 205 are repeated until a predetermined condition is met.

In some embodiments, the pre-determined condition can be set by the user or a computer program automatically. For example, the pre-determined condition can be a time duration for the meditation session. As another example, the pre-determined condition may be a percentage of change of one or more sensed parameters indicating a level of relation in the user. As yet another example, the pre-determined condition can be a variation in a vital sign of the individual.

As a non-limiting example, a VR-enhanced quantitative meditation session can be proceeded by about a 2 minute explanation which can lay out the goals of meditation session(s) and how a user can achieve demonstrable results in a short period of time. Post experience interview lasts about a 2 minute to verify preferences, review change during the meditation experience with the user and optionally highlight reactions to key moments during the intro/outro by the user. An exemplary meditation session plan is shown in Table 1.

TABLE 1

| TIME | ACTIVITY | GOAL |
|---|---|---|
| 2.5 min or | Intro/Preview | Impactful visuals |
| from about | | and environment |
| 1 min to | | preview using the |
| about 5 min | | display |
| 30 sec or | Environment | Gather preferential |
| from about | selection (3-5 | data of the user |
| 20 sec to | different | |
| about 1 min | scenes) | |
| 5 min or | Meditation | Reduce heart rate/blood |
| from about | | pressure of the user |
| 1 min to | | |
| about 30 min | | |
| 30 sec or | Outro/Journey | Impactful closing visuals |
| from about | back to Hale | |
| 20 sec to | | |
| about 1 min | | |
| 8.5 min or | TOTAL TIME | |
| from about | | |
| 2 min to | | |
| about 40 min | | |

As an example, the Intro/Preview of a meditation session is configured to create a visually impactful opening sequence for the users that transports them through the environments that will be featured in the breathing exercise. In some embodiments, such portion of a meditation session can include using the digital display for transitioning the user from an environment projected into the headset through the forward facing cameras to a darkened non-descript expanse. In this particular embodiment, a seed can appear in front of them and start to grow in time lapse. The seed can continue to grow into a full-sized tree. Then another tree can grow and another which can eventually grow to be the entire forest environment. From there the guest can be transported to preview the other virtual environments. In this embodiment, the audio output can be music and ambient sound effects.

In some embodiments, a user can then select an environment among different environments that he/she can perform their meditation in. Selection can be look based (e.g., focusing on an environment for about 6-10 sec to select) and a preview of the environment can appear around the user when they focus on a selection. Audio can change to match the ambience for a given environment as it is selected. User interface audio can also be included to indicate that a user is hitting a selection box. In some embodiments, the meditation portion of each meditation session can have variable durations. For example, a 5-minute meditation session (or 10 minute session, or 20 minute session, or 30 minute session, or 45 minute session, or 60 minute session) can be focused on reducing heart rate, heart rate variability (HRV), or other biometric parameters (such as body posture, pupil dilation, skin surface moisture level, brain wave or brain activity, temperature, blood pressure or any combination thereof). In some embodiments, HRV shows the most marked change during a meditation session as disclosed herein. In some embodiments, meditation can focus on rhythmic breathing. In some embodiments, the virtual environment includes visual and/or audio representations for breathing that help guide the user. In some embodiments, the virtual environment can include visual and/or audio representations such as matching wave action on the beach, movement of trees in the forest, etc., to guide the user in his/her rhythmic breathing. In some embodiments, the movement in the virtual environment is tied to the breathing rhythm of the user. Subsequent to the meditation portion of a session, outro can bridge from the meditation experience and bring the user back into a real environment. As an example, the meditation environment fades out to be replaced with a starry night sky. The user can be in that environment for about 20 seconds and then transition back to the visual representation projected into the headset from the forward facing camera. In some embodiments, the visual representation in VR matches what the user sees when they remove the headset. Use of one or more forward facing cameras may enhance a user's experience of the methods described herein, for example, when transitioning into and out of a virtual reality. The use of one or more forward facing cameras may create a seamless or near-seamless transition into and out of the virtual reality. One or more virtual reality inputs may be provided in a virtual reality. One or more virtual reality inputs may be provided in an augmented reality or mixed reality environment, such as one using video from a forward facing camera in conjunction with overlayed computer imagery.

### User Applications and Controller Applications

In some embodiments, the system includes a user application configured to allow a user to communicate with the remote server. In some embodiments, the application is a mobile application or a web application.

In some embodiments, the application allows the user to view/edit information related to a current meditation session, existing or future meditation sessions. For example, the user can monitor one or more parameters of the user during a meditation session, such as a vital sign. As another example, the user can set a vital sign threshold so that the application sends an audio or mechanical signal when the vital sign exceeds the threshold. In some embodiments, the user can use the application to record the vital sign during a meditation session. In some embodiments, the application allows the user to control one or more elements of the system. In some embodiments, the application allows the user to turn on or turn off one or more sensors, the digital display, and/or the audio output device. In some embodiments, application allows the user to enter additional information related to the meditation session(s) or the user. For example, the application may allow the user to input medical history of the user. As another example, the application may allow the user to input descriptions of his or her symptom(s).

In some embodiments, the application may allow the user to receive a guidance related to adjustment of a meditation session from a remote server or otherwise a digital processing device. In some embodiments, the guidance may include one or more of: an audio signal, a graphical image, a text message, or a combination thereof. In some embodiments, the guidance may include a series of sub-guidances that can be delivered at different time points. In some embodiments, the guidance may be interactive with the user. For example, the one or more sub-guidance may be altered based on the user's response or updated inputs related to the individual to optimize the effect of the meditation session on the user.

In some embodiments, the user application herein allows an individual to view or edit information related to current, existing, and/or future meditations. For example, an individual can view sensed parameter(s) before and after a meditation session to review quantitative effectiveness of the meditation. As another example, the individual may review historical data of the sensed parameter(s) to examine long-term effects of virtual reality (VR)-enhanced quantitative meditations. In some embodiments, the individual can select one or more preferred sensor set-up(s) for measuring one or more parameters. For example, a user may select a body temperature sensor to be attached to his or her body. In some embodiments, the user can enter medical history, symptoms, location of symptoms or other information using the application. In some embodiments, the individual can schedule meditation session(s) using the application.

In some embodiments, the system includes a controller application configured to allow a controller to communicate with the remote server. In some embodiments, the application is a mobile application or a web application.

In some embodiments, the application allows the controller to view/edit information related to a current meditation, existing or future meditation sessions. In some embodiments, the controller can use the application to record vital signs during the current meditation session. In some embodiments, the application allows the controller to control one or more elements of the system. In some embodiments, the application allows the controller to turn on or turn off one or more sensors. In some embodiments, application allows the controller to enter additional information related to the meditation session or the individual.

In some embodiments, a guide/controller can be interfacing via the controller application to the system disclosed herein. One or more of the following information can be displayed in the application: i) where the user currently is in the experience, e.g., intro, selection, meditation, etc, real time biometric data; ii) details about the user, e.g., demographic data; iii) once environment selection is complete, it can be displayed to the controller; iv) at the end of the session, the application can display the results of the biometric data so that the controller can review it with the user, after which the data can be pushed back to a remote server for storage and can be saved locally in the application. Alternatively, the data could be streamed in real time to a data warehouse.

### Machine Learning Algorithms

In some embodiments, the sensed parameter(s) herein is received as an input to output a correlation by a processor. In some embodiments, correlation herein is received as an input to a machine learning algorithm configured to output a guidance or instruction for future meditation sessions and/or future presentation of sensory effect to the user for enhancement of the meditation sessions. In some embodiments, the machine learning algorithm takes additional input(s) in order to output a guidance. In some embodiments, the additional input(s) include description of symptoms by the individual. In some embodiments, the additional input(s) include medical history of the individual. In some embodiments, the additional input(s) includes a medical professional's description of the individual's problem. In some embodiments, the machine learning algorithm is trained and used to output a guidance when an input is received. In some embodiments, the machine algorithm is used to output a guidance while training can be performed before an input is received, for example, periodically using historical data of the individual and/or a selected group of individuals.

The systems, methods, and media described herein may use machine learning algorithms for training prediction models and/or making predictions of a guidance. Machine learning algorithms herein may learn from and make predictions on data. Data may be any input, intermediate output, previous outputs, or training information, or otherwise any information provided to or by the algorithms.

A machine learning algorithm may use a supervised learning approach. In supervised learning, the algorithm can generate a function or model from training data. The training data can be labeled. The training data may include metadata associated therewith. Each training example of the training data may be a pair consisting of at least an input object and a desired output value. A supervised learning algorithm may require the user to determine one or more control parameters. These parameters can be adjusted by optimizing performance on a subset, for example a validation set, of the training data. After parameter adjustment and learning, the performance of the resulting function/model can be measured on a test set that may be separate from the training set. Regression methods can be used in supervised learning approaches.

A machine learning algorithm may use an unsupervised learning approach. In unsupervised learning, the algorithm may generate a function/model to describe hidden structures from unlabeled data (e.g., a classification or categorization that cannot be directed observed or computed). Since the examples given to the learner are unlabeled, there is no evaluation of the accuracy of the structure that is output by the relevant algorithm. Approaches to unsupervised learning include: clustering, anomaly detection, and neural networks.

A machine learning algorithm may use a semi-supervised learning approach. Semi-supervised learning can combine both labeled and unlabeled data to generate an appropriate function or classifier.

A machine learning algorithm may use a reinforcement learning approach. In reinforcement learning, the algorithm can learn a policy of how to act given an observation of the world. Every action may have some impact in the environment, and the environment can provide feedback that guides the learning algorithm.

A machine learning algorithm may use a transduction approach. Transduction can be similar to supervised learning but does not explicitly construct a function. Instead, tries to predict new outputs based on training inputs, training outputs, and new inputs.

A machine learning algorithm may use a “learning to learn” approach. In learning to learn, the algorithm can learn its own inductive bias based on previous experience.

A machine learning algorithm is applied to patient data to generate a prediction model. In some embodiments, a machine learning algorithm or model may be trained periodically. In some embodiments, a machine learning algorithm or model may be trained non-periodically.

As used herein, a machine learning algorithm may include learning a function or a model. The mathematical expression of the function or model may or may not be directly computable or observable. The function or model may include one or more parameter(s) used within a model. For example, a linear regression model having a formula Y=C0+C1x1+C2x2 has two predictor variables, x1 and x2, and coefficients or parameter, C0, C1, and C2. The predicted variable in this example is Y. After the parameters of the model are learned, values can be entered for each predictor variable in a model to generate a result for the dependent or predicted variable (e.g., Y).

In some embodiments, a machine learning algorithm comprises a supervised or unsupervised learning method such as, for example, support vector machine (SVM), random forests, gradient boosting, logistic regression, decision trees, clustering algorithms, hierarchical clustering, K-means clustering, or principal component analysis. Machine learning algorithms may include linear regression models, logistical regression models, linear discriminate analysis, classification or regression trees, naive Bayes, K-nearest neighbor, learning vector quantization (LVQ), support vector machines (SVM), bagging and random forest, boosting and Adaboost machines, or any combination thereof. A machine learning algorithm may perform supervised learning. A machine learning algorithm may perform unsupervised learning. A machine learning algorithm may perform semi-supervised learning. A machine learning algorithm may perform reinforcement learning. Methods may include hierarchical clustering. A machine learning algorithm may be trained with a training set. A training set may comprise training data stored in a database. A training set may comprise measured values of one or more biometric parameters, one or more recipes of a mediation session or any combination thereof. A training set may comprise training data from more than one user. A training set may comprise training data from a single user.

Data input into a machine learning algorithm may include (a) virtual reality input parameters, such as visual and auditory parameters, (b) biometric parameters obtained from an individual receiving the virtual reality parameters—the biometric parameters may be correlated with one or more virtual reality user parameters, (c) additional data such a personal identifying information related to one more individuals, a medical diagnosis, a medical history, a lab metric, a pathology report, or (d) any combination thereof. Biometric parameters input to a machine learning algorithm may be provided by the user, provided by another individual, or provided directly by a sensor that may have obtained the biometric parameter. Virtual reality user parameters may be input to a machine learning algorithm via a user settings or user profile.

Data obtained from one or more mediation sessions (including virtual reality stimulus parameters, individual body metrics, or a combination thereof) can be analyzed using feature selection techniques including filter techniques which may assess the relevance of one or more features by looking at the intrinsic properties of the data, wrapper methods which may embed a model hypothesis within a feature subset search, and embedded techniques in which a search for an optimal set of features may be built into a machine learning algorithm. A machine learning algorithm may identify a set of virtual reality input parameters that may provide an optimized stress reduction or mediation experience for an individual.

A machine learning algorithm may be trained with a training set of samples. The training set of samples may comprise data collected from a mediation session, from different mediation sessions, or from a plurality of mediation sessions. A training set of samples may comprise data from a database.

A training set of samples may include different data types—such as one or more input parameters and one or more output parameters. The input parameters may be an input stimulus provided to an individual and the output parameter may be a biometric response by the individual receiving or not receiving the input stimulus. The input stimulus may be a virtual reality input. A virtual reality input may include visual element, an audio element, or both. A virtual reality input may include a sound type (e.g., classic, jazz, rock, etc), a sound tempo (e.g., fast, slow), a sound volume, a color of light, a light brightness, a rate of change in light color or brightness, a particular scene (e.g., beach, rainforest, clouds, rainbow, flowing water, etc), a song or word phrase (e.g., mantra or poem), or any combination thereof. An individual response or biometric response may include a heart rate, a heart rate variability, a blood pressure, a blood oxygenation level, a breathing pattern, a breathing pace, a neural activity, a skin temperature, a level of perspiration, an eye dilation, a muscle rigidity, a change in any of these, or any combination thereof. An output parameter may be measured as a change in a biometric response from (i) before an input stimulus is provided to (ii) during input stimulation or after the input stimulus is provided, or a combination thereof.

A training set of samples may include about: 1, 2, 3, 4, 5, 6, 7, 8, 9, 10, 11, 12, 13, 14, 15, 20 or more data types. A training set of samples may comprise a single data type. A training set of samples may include different data types. A training set of samples may comprise a plurality of data types. A training set of samples may comprise at least three data types. A training set of samples may include data obtained from about: 1, 2, 3, 4, 5, 6, 7, 8, 9, 10, 11, 12, 13, 14, 15, 20 or more individuals. A training set of samples may include data obtained from about 1 to about 20 individuals. A training set of samples may include data obtained from about 1 to about 100 individuals. A training set of samples may include data obtained from about 1 to about 200 individuals. A training set of samples may include data from a single individual. A training set of samples may include data from different individuals. A training set of samples may include data from a plurality of individuals.

Iterative rounds of training may occur to arrive at a set of features to classify data. Different data types may be ranked differently by the machine learning algorithm. One data type may be ranked higher than a second data type. Weighting or ranking of data types may denote significance of the data type. A higher weighted data type may provide an increased accuracy, sensitivity, or specificity of the classification or prediction of the machine learning algorithm. For example, an input parameter of sound tempo (of a virtual reality scene) may significantly reduce blood pressure, more than any other input parameter. In this case, sound tempo may be weighted more heavily than other input parameters in reducing blood pressure. The weighting or ranking of features may vary from individual to individual. The weighting or ranking of features may not vary from individual to individual.

A machine learning algorithm may be tested with a testing set of samples. The testing set of samples may be different from the training set of samples. At least one sample of the testing set of samples may be different from the training set of samples. The testing set of samples may comprise data collected from before a mediation, from a mediation session, from different mediation sessions, or from a plurality of mediation sessions. A testing set of samples may comprise data from a database.

A training set of samples may include different data types—such as one or more input parameters and one or more output parameters. An input parameter may include a virtual reality input—such as a sound type (e.g., classic, jazz, rock, etc), a sound tempo (e.g., fast, slow), a sound volume, a color of light, a light brightness, a rate of change in light color or brightness, a particular scene (e.g., beach, rainforest, clouds, rainbow, flowing water, etc), a song or word phrase (e.g., mantra or poem), or any combination thereof. An output parameter may include a heart rate, a heart rate variability, a blood pressure, a blood oxygenation level, a breathing pattern, a breathing pace, a neural activity, a skin temperature, a level of perspiration, an eye dilation, a muscle rigidity, a change in any of these, or any combination thereof. A testing set of samples may include about: 1, 2, 3, 4, 5, 6, 7, 8, 9, 10, 11, 12, 13, 14, 15, 20 or more data types. A testing set of samples may include from about 1 data type to about 5 data types. A testing set of samples may include from about 1 data type to about 10 data types. A testing set of samples may include from about 1 data type to about 20 data types. A testing set of samples may comprise a data type. A testing set of samples may include different data types. A testing set of samples may comprise a plurality of data types. A testing set of samples may comprise at least three data types. A testing set of samples may include data obtained from about: 1, 2, 3, 4, 5, 6, 7, 8, 9, 10, 11, 12, 13, 14, 15, 20 or more individuals. A testing set of samples may include data obtained from about 1 individual to about 5 individuals. A testing set of samples may include data obtained from about 1 individual to about 10 individuals. A testing set of samples may include data obtained from about 1 individual to about 20 individuals. A testing set of samples may include data from a single individual. A testing set of samples may include data from different individuals. A testing set of samples may include data from a plurality of individuals.

A machine learning algorithm may classify or predict an outcome with at least about: 80%, 85%, 90%, 95%, 96%, 97%, 98%, 99% accuracy. An algorithm may classify an outcome with an accuracy from about 90% to 100%. An algorithm may classify an outcome with an accuracy from about 95% to 100%. An algorithm may classify an outcome with an accuracy from about 96% to 100%. A machine learning algorithm may classify or predict an outcome with at least about: 80%, 85%, 90%, 95%, 96%, 97%, 98%, 99% sensitivity. An algorithm may classify an outcome with a sensitivity from about 90% to 100%. An algorithm may classify an outcome with a sensitivity from about 95% to 100%. An algorithm may classify an outcome with a sensitivity from about 96% to 100%. A machine learning algorithm may classify or predict an outcome with at least about: 80%, 85%, 90%, 95%, 96%, 97%, 98%, 99% specificity. An algorithm may classify an outcome with a specificity from about 90% to 100%. An algorithm may classify an outcome with a specificity from about 95% to 100%. An algorithm may classify an outcome with a specificity from about 96% to 100%. For example, a machine learning algorithm may classify with about 90% accuracy that one or more virtual reality inputs may produce a change in one or more biometric parameters in an individual receiving the one or more virtual reality inputs. A machine learning algorithm may classify an individual as having at least about 90% likelihood of a stress reduction after receiving a virtual reality input. The stress reduction may be measured by one or more biometric parameters. A machine learning algorithm may predict at least 95% likelihood of increased relaxation in an individual after receiving a set of virtual reality input parameters.

An independent sample may be independent from the training set of samples, the testing set of samples or both. The independent sample may be input into the machine learning algorithm for classification. An independent sample may not have been previously classified by the machine learning algorithm.

A classifier may be employed to determine or to predict a set of virtual reality parameters to be administered to the individual, such as to reduce a stress or induce a relaxation in the individual. A classifier may be employed to predict a change in one or more biometric parameters of an individual that may receive a set of virtual reality parameters. A classifier may provide real-time feedback and guided adjustments of the one or more virtual reality parameters to optimize one or more biometric parameters—such as during a mediation session. One or more virtual reality parameters may be adjusted real-time during a mediation session based on a biometric parameter of an individual.

Use of a machine learning algorithm may promote or optimize relaxation or reduce stress in an individual receiving a virtual reality input based on the one or more biometric parameters obtained from the individual. A machine learning algorithm may identify an ‘ideal’ or ‘optimized’ input parameter for each individual. An ‘ideal’ or ‘optimized’ input parameter may remain constant or may change over time. An ‘ideal’ or ‘optimized’ input parameter may be specific or unique for each individual. Feedback from a machine learning algorithm may be continuous such as feedback during a mediation session, episodic such as at the end of a mediation session, or roll-back such as cumulative changes over several different sessions, or any combination thereof. Feedback from a machine learning algorithm may result in one or more changes in a virtual reality input. For example, feedback from a machine learning algorithm may adjust a sound volume, a sound type, a scene, a brightness of light, or any other virtual reality input.

### Digital Processing Device

In some embodiments, the platforms, systems, media, and methods described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected to a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a digital display to send visual information to a user.

In some embodiments, the digital processing device includes an input device to receive information from a user. In some embodiments, the input device is a keyboard. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

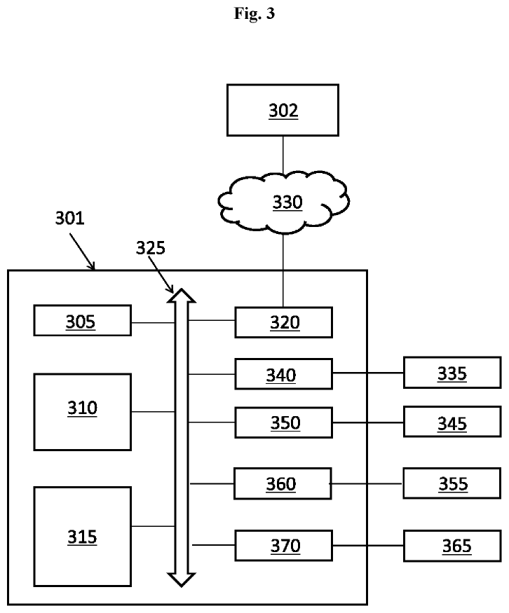

Referring to FIG. 3, in a particular embodiment, an exemplary digital processing device 301 is programmed or otherwise configured to control sensing, sensing data communication, sensing data processing, and generation of correlation data of the sensed parameter and the sensory presentation to the user using systems and methods herein. In this embodiment, the digital processing device 301 includes a central processing unit (CPU, also “processor” and “computer processor” herein) 305, which can be a single core or multi core processor, or a plurality of processors for parallel processing. The digital processing device 301 also includes memory or memory location 310 (e.g., random-access memory, read-only memory, flash memory), electronic storage unit 315 (e.g., hard disk), communication interface 320 (e.g., network adapter, network interface) for communicating with one or more other systems, and peripheral devices, such as cache, other memory, data storage and/or electronic display adapters. The peripheral devices can include storage device(s) or storage medium 365 which communicate with the rest of the device via a storage interface 370. The memory 310, storage unit 315, interface 320 and peripheral devices are in communication with the CPU 305 through a communication bus 325, such as a motherboard. The storage unit 315 can be a data storage unit (or data repository) for storing data. The digital processing device 301 can be operatively coupled to a computer network (“network”) 330 with the aid of the communication interface 320. The network 330 can be the Internet, an internet and/or extranet, or an intranet and/or extranet that is in communication with the Internet. The network 330 in some embodiments is a telecommunication and/or data network. The network 330 can include one or more computer servers, which can enable distributed computing, such as cloud computing. The network 330, in some embodiments—with the aid of the device 301, can implement a peer-to-peer network, which can enable devices coupled to the device 301 to behave as a client or a server.

Continuing to refer to FIG. 3, the digital processing device 301 includes input device(s) 345 to receive information from a user, the input device(s) in communication with other elements of the device via an input interface 350. The digital processing device 301 can include output device(s) 355 that communicates to other elements of the device via an output interface 360.

Continuing to refer to FIG. 3, the memory 310 can include various components (e.g., machine readable media) including, but not limited to, a random-access memory component (e.g., RAM) (e.g., a static RAM “SRAM”, a dynamic RAM “DRAM, etc.), or a read-only component (e.g., ROM). The memory 310 can also include a basic input/output system (BIOS), including basic routines that help to transfer information between elements within the digital processing device, such as during device start-up, can be stored in the memory 310.

Continuing to refer to FIG. 3, the CPU 305 can execute a sequence of machine-readable instructions, which can be embodied in a program or software. The instructions can be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and write back. The CPU 305 can be part of a circuit, such as an integrated circuit. One or more other components of the device 301 can be included in the circuit. In some embodiments, the circuit is an application specific integrated circuit (ASIC) or a field programmable gate array (FPGA).

Continuing to refer to FIG. 3, the storage unit 315 can store files, such as drivers, libraries and saved programs. The storage unit 315 can store user data, e.g., user preferences and user programs. The digital processing device 301 in some embodiments, can include one or more additional data storage units that are external, such as located on a remote server that is in communication through an intranet or the Internet. The storage unit 315 can also be used to store operating systems, application programs, and the like. Optionally, storage unit 315 can be removably interfaced with the digital processing device (e.g., via an external port connector (not shown)) and/or via a storage unit interface. Software may reside, completely or partially, within a computer-readable storage medium within or outside of the storage unit 315. In another example, software may reside, completely or partially, within processor(s) 305.

Continuing to refer to FIG. 3, the digital processing device 301 can communicate with one or more remote computer systems 302 through the network 330. For instance, the device 301 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants. In some embodiments, the remote computer system is configured for image and signal processing of images acquired using the image systems herein. In some embodiments, the imaging systems herein allows partitioning of image and signal processing between a processor in the imaging head (e.g. based on a MCU, DSP or FPGA) and a remote computer system, e.g., a back-end server.

Continuing to refer to FIG. 3, information and data can be displayed to a user through a display 335. The display is connected to the bus 325 via an interface 340, and transport of data between the display other elements of the device 301 can be controlled via the interface 340.

Methods as described herein can be implemented by way of machine (e.g., computer processor) executable code stored on an electronic storage location of the digital processing device 301, such as, for example, on the memory 310 or electronic storage unit 315. The machine executable or machine-readable code can be provided in the form of software. During use, the code can be executed by the processor 305. In some embodiments, the code can be retrieved from the storage unit 315 and stored on the memory 310 for ready access by the processor 305. In some situations, the electronic storage unit 315 can be precluded, and machine-executable instructions are stored on memory 310.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some embodiments, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

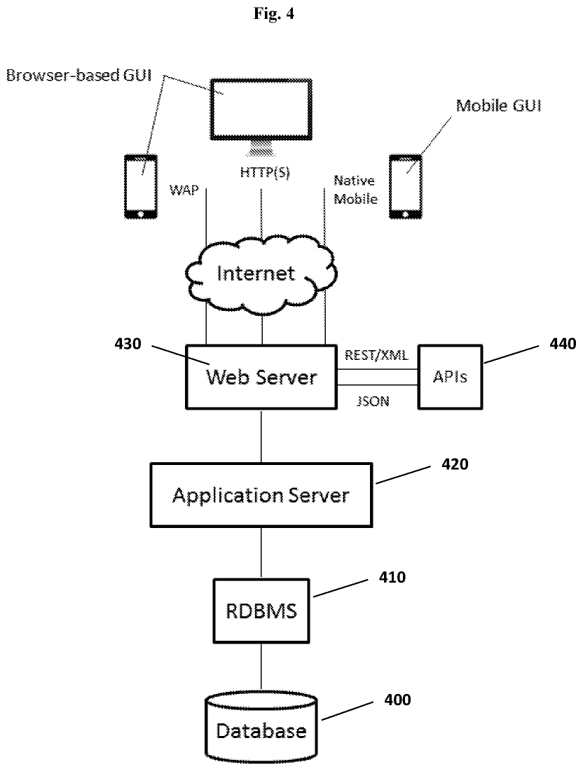

Referring to FIG. 4, in a particular embodiment, an application provision system comprises one or more databases 400 accessed by a relational database management system (RDBMS) 410. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 420 (such as Java servers, .NET servers, PHP servers, and the like) and one or more web servers 430 (such as Apache, IIS, GWS and the like). The web server(s) optionally expose one or more web services via app application programming interfaces (APIs) 440. Via a network, such as the Internet, the system provides browser-based and/or mobile native user interfaces.

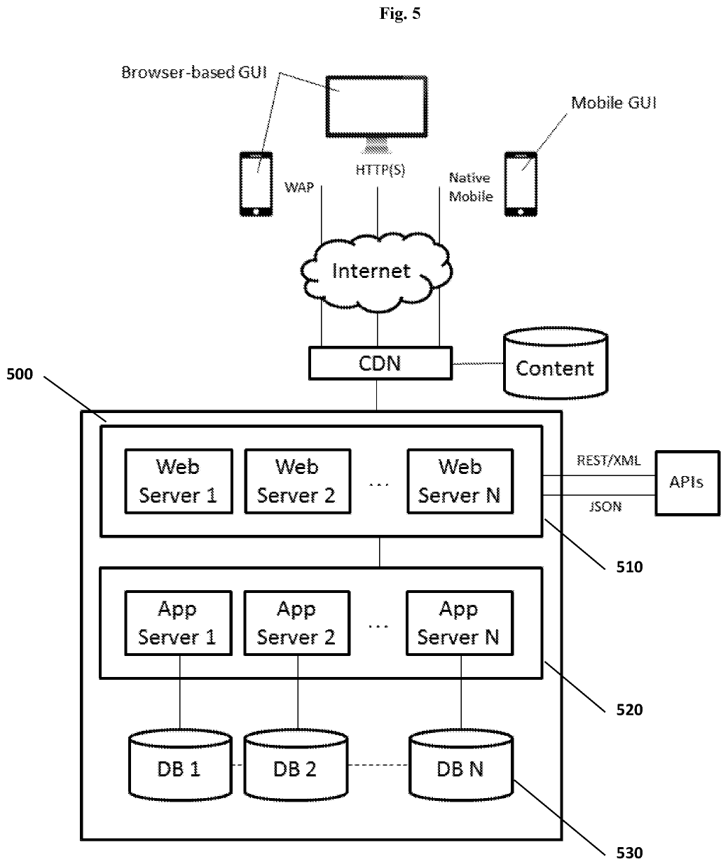

Referring to FIG. 5, in a particular embodiment, an application provision system alternatively has a distributed, cloud-based architecture 500 and comprises elastically load balanced, auto-scaling web server resources 510 and application server resources 520 as well synchronously replicated databases 530.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of meditation record, information of a user (e.g., demographic information), parameters, sensing data, and/or correlation data. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

While preferred embodiments have been shown and described herein, it will be obvious to those skilled in the art that such embodiments are provided by way of example only. Numerous variations, changes, and substitutions will now occur to those skilled in the art without departing from the scope of the disclosure. It should be understood that various alternatives to the embodiments described herein may be employed in practice. Numerous different combinations of embodiments described herein are possible, and such combinations are considered part of the present disclosure. In addition, all features discussed in connection with any one embodiment herein can be readily adapted for use in other embodiments herein. It is intended that the following claims define the scope of the disclosure and that methods and structures within the scope of these claims and their equivalents be covered thereby.

What is claimed is:

1. A system for use with a meditation session, said system comprising:
(a) a virtual reality display configured to display a virtual environment comprising a plurality of virtual images to a user while the user meditates;
(b) a biometric sensor configured to sense a plurality of biometric parameters of said user; and
(c) a processor configured to correlate said virtual environment or at least one virtual image of said plurality of virtual images with at least one biometric parameter of said plurality of biometric parameters.

2. The system of claim 1, wherein said display is a head mounted display.

3. The system of claim 1, wherein each of the plurality of virtual images comprises a portion of said virtual environment.

4. The system of claim 3, wherein the virtual environment comprises a scene from nature.

5. The system of claim 1, wherein the biometric sensor comprises a heart rate sensor, a blood pressure sensor, or an spO₂ sensor.

6. The system of claim 5, wherein the biometric sensor is used to determine at least one of a heart rate variability or a respiratory rate.

7. The system of claim 1, wherein the system further comprises: (d) an audio output device configured to provide a plurality of audio outputs to the user.

8. The system of claim 7, wherein the at least one of the plurality of audio outputs corresponds to at least one of the plurality of virtual images.

9. The system of claim 7, wherein the processor is further configured to correlate at least one of said audio outputs with at least one of said plurality of biometric parameters.

10. The system of claim 9, wherein the processor is further configured to cause said audio output device to again output said at least one of said audio outputs during said meditation session or a subsequent meditation session.

11. The system of claim 1, wherein the processor is further configured to cause said virtual reality display to display said at least one virtual image to said user during said meditation session or a subsequent meditation session.

12. The system of claim 1, wherein the processor is further configured to cause said virtual reality display to again display said at least one virtual image to said user when said at least one biometric parameter is a certain value.

13. The system of claim 12, wherein said biometric sensor comprises a heart rate sensor and said certain value is a heart rate less than about 70 beats per minute (bpm).

14. The system of claim 12, wherein said biometric sensor comprises a blood pressure sensor and said certain value is a systolic blood pressure less than about 130 mmHg.

15. The system of claim 1, wherein the processor is further configured to cause said virtual reality display to again display said at least one virtual image to said user during said meditation session or a subsequent meditation session when said at least one biometric parameter is less than a baseline biometric parameter of the user that is previously sensed.

16. A method for use during a meditation session, said method comprising:
(a) displaying a virtual environment comprising a plurality of virtual images to a user while the user meditates;
(b) sensing a plurality of biometric parameters of said user; and
(c) correlating, with a processor, said virtual environment or at least one virtual image of said plurality of virtual images with at least one biometric parameter of said plurality of biometric parameters.

17. A non-transitory computer readable storage medium comprising computer readable software configured to cause a processor to:
(d) display a virtual environment comprising a plurality of virtual images to a user while the user meditates;
(e) sense a plurality of biometric parameters of said user; and
(f) correlate said virtual environment or at least one virtual image of said plurality of virtual images with at least one biometric parameter of said plurality of biometric parameters.

18. A computer implemented method for providing a meditation session, said method comprising:
(a) sensing at least one parameter from an individual while said individual is meditating during a meditation session;
(b) inputting the at least one parameter into a machine learning software module; and
(c) determining, with the machine learning software module, a modification of the meditation session.

19. The method of claim 18, comprising displaying, with a display, a virtual environment comprising a plurality of virtual images to a user while the user meditates.

20. The method of claim 19, wherein said display is a head mounted display.

21. The method of claim 19, wherein each of the plurality of virtual images comprises a portion of said virtual or augmented environment.

22. The method of claim 21, wherein the virtual environment comprises a scene from nature.

23. The method of claim 18, wherein the at least one parameter comprises a heart rate, a blood pressure, or an spO₂.

24. The method of claim 18, wherein the at least one parameter comprises at least one of a heart rate variability or a respiratory rate.

25. The method of claim 18, comprising outputting a plurality of audio outputs to the individual.

26. The method of claim 25, wherein the at least one of the plurality of audio outputs corresponds to at least one of a plurality of virtual images.

27. The method of claim 26, comprising correlating at least one of said audio outputs with the at least one parameter.

28. The method of claim 27, wherein the processor is further configured to cause said audio output device to again output said at least one of said audio outputs during said meditation session or a subsequent meditation session.
